(19) United States
(12) Patent Application Publication
(10) Pub. No.: US 2023/0277701 A1
(43) Pub. Date: Sep. 7, 2023

(54) DEVICE FOR ACCOMODATING AND DISINFECTING A WRITING INSTRUMENT

(71) Applicant: RONOTIC AG, St. Leon-Rot (DE)

(72) Inventor: Cem Koc, Walldorf (DE)

(21) Appl. No.: 18/006,665

(22) PCT Filed: Jul. 28, 2021
(86) PCT No.: PCT/EP2021/000087

§ 371 Date: Jan. 24, 2023

(30) Foreign Application Priority Data
Jul. 28, 2020 (DE) 10 2020 004 845.5
Jul. 28, 2020 (DE) 20 2020 003 412.6
Oct. 14, 2020 (DE) 10 2020 006 483.3
Oct. 14, 2020 (DE) 20 2020 004 428.8

(51) Int. Cl. A61L 2/10; A61L 2/24

(52) U.S. Cl. CPC A61L 2/10; A61L 2/24; A61L 2202/11; A61L 2202/121; A61L 2202/122; A61L 2202/14; A61L 2202/20

(57) ABSTRACT

The invention relates to a device for accommodating and disinfecting a writing instrument (4), comprising at least a housing (1) having a chamber with at least one opening for placing or inserting the writing instrument (4) in the chamber, the chamber being an illumination chamber (2) equipped with UV light sources (3) that irradiate the entire outer surface of the placed or inserted writing instrument (4).

In a first aspect of the invention, the same relates to a device for accommodating and disinfecting at least one writing instrument which is primarily used in public institutions, banks, etc. by the public.

Accommodators for writing instruments in the form of grommets or plates provided with insertion openings are known. The writing instruments are, inter alia, taken out for the purpose of signatures as well as for filling out forms and then inserted back into the accommodator.

This means that a plurality of persons is in contact with the writing instruments without them being subjected to disinfection after usage. In the best case, they are wiped over at intervals with a cloth or piece of paper soaked in disinfectant.

This is also the case for the trough-shaped accommodator for writing instruments described in DE 1 799 647 U. In this case, the accommodator comprises the feature of a lateral indentation in order to grasp the respective writing instrument more easily.

Considering the risk of spreading bacteria, viruses, and germs due to multiple uses, it is the object of the invention of the first aspect of the invention to disinfect writing instruments used by different persons after each usage.

Thus, it is a clear objective of the invention to conduct a disinfection of a frequently used writing instrument used by different persons.

This object is achieved with the features of Claim 1. Advantageous configurations are the subject matter of the subclaims. A particularly preferable embodiment according to the invention is the trough-shaped recess of the device.

Therefore, an achievement of the object is, for example, the optimal positioning of the writing instrument in the device according to the invention in such a way that the writing instrument is aligned optimally opposite the UV light sources for the writing instrument to be sufficiently disinfected by irradiation, optionally with a suitable minimum duration and irradiation intensity.

The use of UV light, in particular UVC light, for disinfection is fundamentally known when it comes to bacteria, germs, and viruses.

At the same time, however, an excessive irradiation of the human hand with such UV rays is harmful, giving rise to a first conflict to be solved.

Furthermore, the device according to the present invention relates to writing instruments. In this case, it is not just any writing instrument, but rather a writing instrument stored in a public space and used by a plurality of persons here: e.g. for filling out bank slips or for filling out application forms in municipal offices.

This gives rise to a second conflict to be solved: The writing instrument must be stored visibly, be graspable by hands of different sizes, and must not be hazardous to the health of the user, in particular due to germ and/or virus loads. In this context, it should also be noted that the UV dosage should not be physiologically hazardous; instead, exposure should be either avoided entirely or at least only present in ranges acceptable health-wise, as, for example, in the case of natural UV radiation.

Furthermore, it makes little sense to only disinfect the writing instrument in a closed container if the user has to reopen the container by hand. This would only shift the site of infection.

The device according to the invention for accommodating and disinfecting a writing instrument, comprising at least a housing having a chamber with at least one opening for placing or inserting the writing instrument in the chamber, provides that the chamber is an illumination chamber equipped with UV light sources, preferably UVC light sources, that irradiate the entire outer surface of the placed or inserted writing instrument.

The illumination chamber is preferably embodied as a trough-shaped recess or trough or as a bore or blind hole.

The light sources may be LEDs or formed to be pipe-shaped.

In order to ensure an irradiation of the writing instrument from the direction of the at least one opening, the device provides for two configurations, which may also be combined.

On the one hand, the illumination chamber formed to be a trough-shaped recess or trough can comprise a depth which is greater than the greatest cross-section of the writing instrument or, in the case of bores or blind holes, the depth is greater than the length of the writing instrument.

In another embodiment, a cover is arranged above the opening for placing the writing instrument or a partial cover is arranged spaced apart from the opening, said partial cover also being fitted with UV light sources, preferably UVC light sources, on the inside. The cover is arranged to be unfoldable or slidable or peelable.

In order to be able to optimally use the UV light for disinfecting, the distance between the light sources and the writing instrument should be the same everywhere. This can be realized by centering elements for the writing instruments in the illumination chamber. For a trough-shaped recess or a trough, these may be, for example, rods or a double fork, as is known from older telephones. Upon placing the writing instrument in the trough-shaped recess or trough, the writing instrument then slides or rolls into the designated central position.

A further advantageous configuration of the device provides that the UV light sources, preferably UVC light sources, are able to be switched on, preferably temporarily, by placing or inserting the writing instrument in the illumination chamber. To this end, preferably at least one movement sensor is arranged on the housing or a pressure switch is arranged in the illumination chamber. The pressure switch should in this case be activatable by the weight of the writing instrument. The function of the pressure switch can in this case also be assumed by one or several of the centering elements.

In this advantageous embodiment, simultaneously, upon placing or inserting the writing instrument in the illumination chamber and activating the light sources, a timer is actuated which switches the light sources off again after a predetermined time.

In a further configuration of the device, it is provided that the illumination chamber formed as a trough-shaped recess or trough comprises at least one cut-out in the housing which is arranged laterally from the placed writing instrument. This makes it easier to grasp the writing instrument.

An embodiment further provides that the housing has a writing support next to the illumination chamber. This writing support can also be a digitally operating one.

The power supply to the light sources and the electronic equipment is preferably realized through rechargeable batteries or a network connection. The latter has the advantage that it is simultaneously a type of attachment and thus acts as an at least minor theft deterrent.

Advertisement, e.g. a company logo, can be arranged on the surface of the housing.

The invention is to be explained using the drawings. Therein:

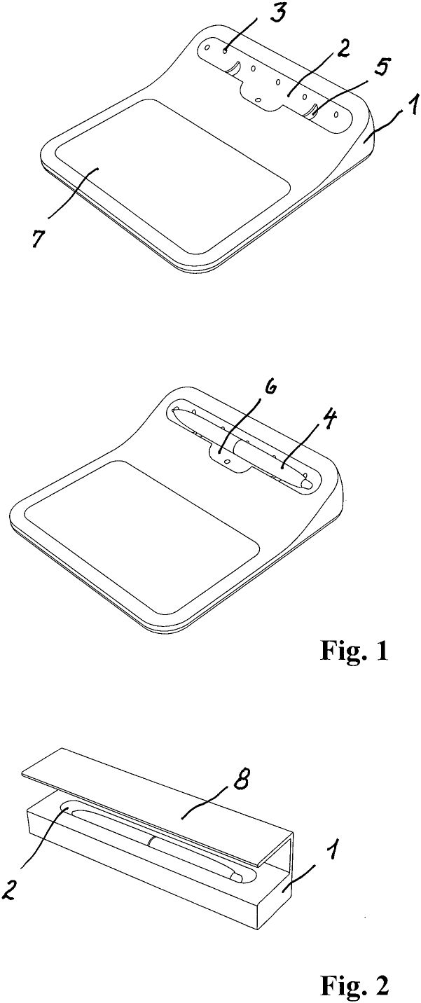

FIG. 1 shows the housing of the device having a trough-shaped recess and a writing support and

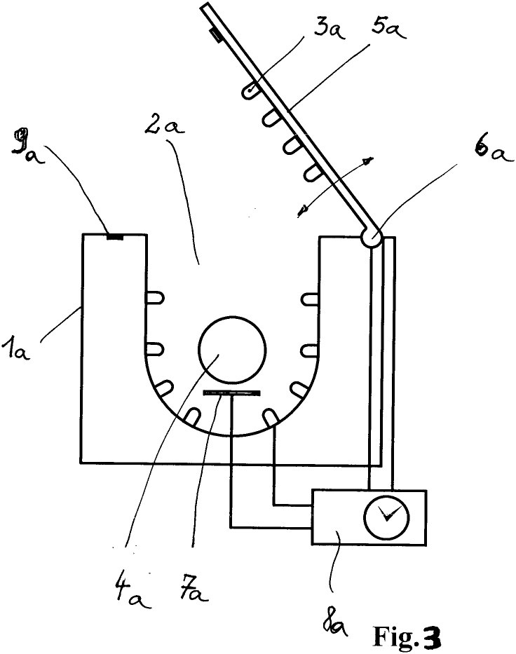

FIG. 2 shows the housing of the device having a trough-shaped recess and a partial cover.

FIG. 1 shows the housing 1 of the device according to the invention for accommodating and disinfecting a writing instrument 4 having a trough-shaped recess of the illumination chamber 2, in one example with a writing instrument and in another without one.

In this embodiment, the housing 1 further comprises a writing support 7. Especially for signatures, a corresponding writing support 7 is advantageous, as it is embodied to be smooth and slip-proof.

Naturally, it is also possible to use such writing supports for digital uses, e.g. also for the signature.

The walls of the trough-shaped recess are fitted with individual UVC light sources 3.

In order to also ensure irradiation with UVC light from above, i.e. where the opening for placing and removing the writing instrument 4 is located, the trough-shaped recess comprises a depth that is greater than the greatest cross-section of the writing instrument 4. Due to the UV light sources located on the upper edge, this surface of the writing instrument 4, too, is irradiated.

In the context of the invention, “irradiated from above” or similar expressions in several embodiments do not necessarily mean that the UV rays, preferably UVC rays, irradiate an outer surface of the writing instrument at a 90° angle or at an essentially 90° angle. Rather, “irradiated from above” or the like may in some embodiments of the invention also comprise a lateral irradiation; i.e. an angle of impact smaller than 90° relative to the outer surface of the writing instrument as the plane of reference.

In one embodiment of the invention, the UV light, preferably the UVC light, consequently irradiates the outer surface of the writing instrument at an appropriate angle of the UV rays, preferably UVC rays, such that they do not radiate perpendicularly, but instead at an angle < 90°, preferably < 80°, more preferably < 70°, even more preferably < 45° relative to the outer surface of the writing instrument.

In order to still allow for an unproblematic removal of the writing instrument from the trough-shaped recess, the trough-shaped recess comprises one or two, preferably opposing, cut-outs 6. This makes it easier to grasp the writing instrument 4 with one’s fingers.

For effective irradiation of the surface writing instrument 4, it is important that the UV light sources, preferably UVC light sources, are the same distance from the surface of the writing instrument 4 essentially everywhere. To this end, centering elements 5, e.g. in the form of pegs or cones, are arranged between the LED light sources 3. This way, the writing instrument 4 assumes the optimal position itself upon being placed in the trough-shaped recess.

If the writing instrument 4 is not used for a while, it need not be constantly disinfected. Therefore, an advantageous configuration provides that the UV light sources 3, preferably UVC light sources, are able to be switched on, preferably temporarily, by placing the writing instrument 4 in the illumination chamber 2.

For switching on the UV light sources 3, preferably UVC light sources, the housing 1 is equipped with at least a movement sensor or a pressure switch. In the representation, the centering element 5 assumes the function of the pressure switch.

The embodiment represented in FIG. 2, also in the form of a trough-shaped recess of the illumination chamber 2, differs from the embodiment described above essentially in two features.

Firstly, above the opening for placing the writing instrument 4, a partial cover 8 is arranged spaced apart from the opening, said partial cover also being fitted with UV light sources 3, preferably UVC light sources, on the inside. Due to the fitting of this partial cover 8 with UV light sources 3, preferably UVC light sources, the surface of the writing instrument 4 pointing in the direction of the opening for placing or removing can be more optimally irradiated than by lateral UV light sources 3, preferably UVC light sources.

The housing 1 further comprises no writing support.

### LIST OF REFERENCE NUMERALS

1 Housing
2 Illumination chamber
3 UV light source
4 Writing instrument
5 Centering elements
6 Cut-out
7 Writing support
8 Partial cover

In consideration of the aforementioned full disclosure of the first aspect of the invention, said aspect further comprises the embodiments as numbered in the following:

1. A device for accommodating and disinfecting a writing instrument (4), comprising at least a housing (1) having a chamber with at least one opening for placing or inserting the writing instrument (4) in the chamber, characterized in that

the chamber is an illumination chamber (2) equipped with UV light sources (3) that irradiate the entire outer surface of the placed or inserted writing instrument (4).

2. The device according to Embodiment 1, characterized in that

the illumination chamber (2) is formed to be a trough-shaped recess or trough or to be a bore or a blind hole.

3. The device according to Embodiment 1 or 2, characterized in that

the UV light sources (3) emit UVC light.

4. The device according to any one of Embodiments 1 to 3, characterized in that

centering elements (5) for the writing element (4) are arranged in the illumination chamber (2).

5. The device according to any one of Embodiments 2 to 4, characterized in that

the illumination chamber (2) formed to be a trough-shaped recess or trough comprises a depth which is greater than the greatest cross-section of the writing instrument (4) and, in the case of a bore or a blind hole, the depth is greater than the length of the writing instrument (4).

6. The device according to any one of Embodiments 1 to 5, characterized in that

a cover is arranged above the opening for placing the writing instrument (4) or a partial cover (8) is arranged spaced apart from the opening, said partial cover also being fitted with UV light sources (3) on the inside.

7. The device according to Embodiment 6, characterized in that

the cover or the partial cover is arranged to be unfoldable or slidable or peelable.

8. The device according to any one of Embodiments 1 to 7, characterized in that

the UV light sources (3), preferably UVC light sources, are able to be switched on, preferably temporarily, by placing or inserting the writing instrument (4) in the illumination chamber (2).

9. The device according to Embodiment 8, characterized in that

in order to switch on the UV light sources (3), preferably UVC light sources, the housing (1) comprises at least a movement sensor or a pressure switch.

10. The device according to Embodiment 9, characterized in that

at least one centering element (5) assumes the function of the pressure switch additionally.

11. The device according to any one of Embodiments 2 to 10, characterized in that

the illumination chamber (2) formed as a trough-shaped recess or trough comprises at least one cut-out (6) in the housing (1) which is arranged laterally from the placed writing instrument (4).

12. The device according to any one of Embodiments 1 to 11 characterized in that

the housing (1) has a writing support (7).

13. The device according to Embodiment 12, characterized in that

the writing support (7) is a digitally operating one.

It has been shown that even in the case of a provided temporary switching on of the UV light sources (as explained above) it is not completely and necessarily ensured that the hand grasping for the writing instrument or placing down the writing instrument is no longer exposed to rays of UV light.

Therefore, it is an object of a second aspect of the invention to eliminate any contact of the human hand with the UV light during the usage of the disinfecting effect of UV light. Furthermore, according to a second aspect of the invention, advantageous configurations of an illumination chamber are to be proposed.

This object is achieved with the features of Claims 8 and 15. Advantageous configurations are the subject matter of the dependent subclaims.

For a device for accommodating and disinfecting a writing instrument, comprising at least a housing having an illumination chamber which is illuminatable with UV light, preferably UVC light, which irradiates the entire outer surface of the writing instrument placed or inserted through an opening, it is proposed according to the invention according to the second aspect of the invention that the illumination chamber is completely closed by a closed cover and the light sources for the UV light, preferably UVC light, is only activatable when the illumination chamber is entirely closed and deactivatable upon or before the opening of the illumination chamber.

According to the second aspect of the invention, the cover is preferably a foldable or slidable or peelable lid or a blind which has an automatic opening and closing mechanism which is touchlessly activatable.

In a specific embodiment, the cover is configured with, electively, a sliding door, folding door or slat door or subcombinations thereof - in the technical sense of certain old secretary-desks. This means that, like blinds or shutters, these latter variations of the covers can be rolled up and rolled out via foldable and/or tiltable individual elements in order to allow for a movable yet sealed closure of the illumination chamber. These embodiments, too, can be combined with a timer in a suitable manner in order to elicit temporary opening and closing states of the illumination chamber.

Due to the automatic opening and closing mechanism, a shift of the infection hazard from the writing instrument to the housing is prevented.

UV light includes UVC light or may be only UVC light.

Two fundamental embodiments according to the second aspect of the invention are applied as the automatic opening and closing mechanism.

The first embodiment according to the second aspect of the invention is associated with a human action. This includes, e.g., a movement sensor arranged on the housing which generates a signal for actuating the automatic opening and closing mechanism through a human action. The movement sensor may also be a light barrier.

An insertion slot for any plastic card having a chip card format may also be provided in the housing, wherein the inserting or removing of the plastic card is detectible and a signal for opening or closing the cover is triggerable therethrough.

The second embodiment according to the second aspect of the invention operates independently of human involvement. In this case, for example, the housing has a time switch which deactivates the UV light sources after a predefinable time of UV light irradiation of the writing instrument and then generates a signal for opening the illumination chamber of the housing such that the writing instrument is accessible.

The person can in this case only access a disinfected writing instrument when the UV light sources are switched off without prior action of the person being necessary.

The closing of the illumination chamber is coupled with the writing instrument in a known manner, such that a signal for closing is generatable upon placing. Possible embodiments are provided in DE 20 2020 003 412 U1.

A further advantageous configuration according to the second aspect of the invention provides that the cover and/or the rest surface of the cover is equipped with a seal on the housing.

The illumination of the illumination chamber with UV light occurs either directly via the light sources and/or through illumination slits which are placed in front of the light sources. In addition to covering, the illumination slits may be used as a seal against UV radiation.

A further advantageous configuration according to the second aspect of the invention provides that the illumination chamber is at least partially lined or coated with UV light-reflecting means. This increases the effectiveness of the UV irradiation and can shorten the duration of the disinfection.

The latter two embodiments according to the second aspect of the invention also lead to an improvement in the disinfection, even independently of the protection against UV light.

In an additional and/or alternative embodiment, the device of the present invention further comprises a removable and/or insertable device, more preferably a telescopic removable and/or insertable rod device which may be electronically and/or mechanically removed and which, like an elevator, can move upward and/or downward and/or laterally inward and/or outward therewith in order to move the comprised writing instrument 4a into the illumination chamber 2a and/or out of the illumination chamber 2a, preferably being electronically controlled (8b) and the light sources (3a) for the UV light only being activatable with a writing instrument (4a) at least partially, preferably entirely, inserted in the illumination chamber (2a) and deactivatable with the writing instrument (4a) at least partially, preferably entirely, removed from the illumination chamber (2a).

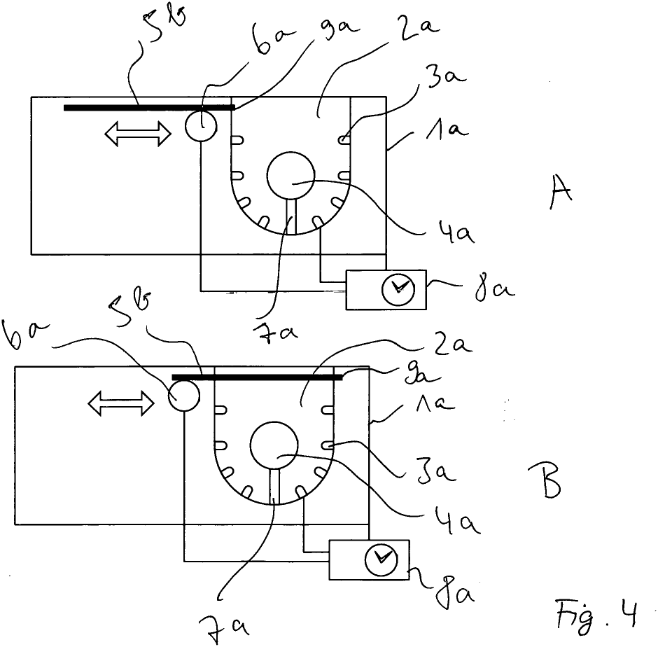

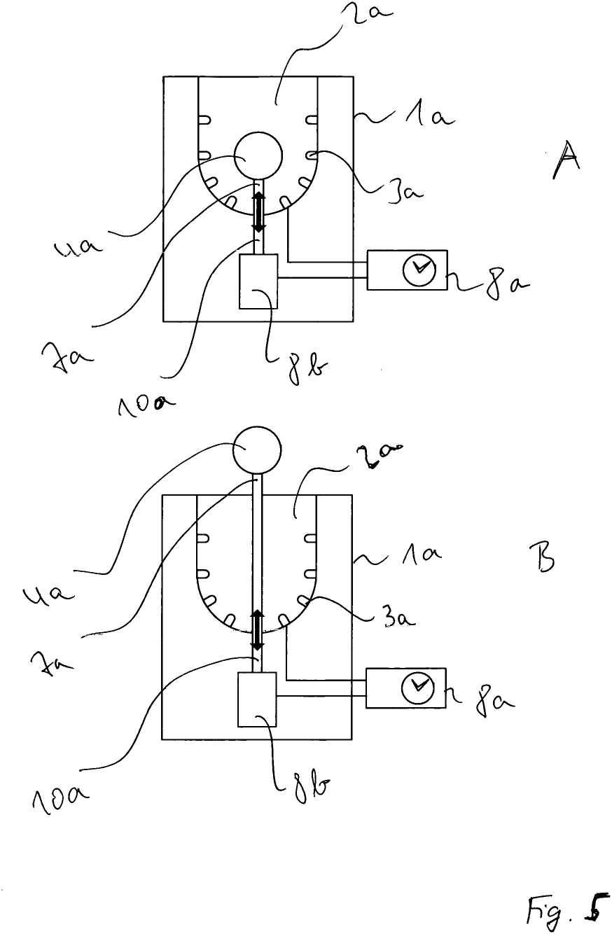

The invention according to Claim 8 is to be, inter alia, explained using the drawing according to FIG. 3. The FIGS. 4 and 5 further show supplemental and/or alternative embodiments.

FIG. 3 shows a tub-like housing 1a for accommodating a writing instrument 4a. The housing 1a is closeable with an unfoldable cover 5a, in this case a lid. Both the inner tub walls of the housing 1a and the cover 5a are fitted with light sources 3a which can radiate UV light or UVC light and thereby form an illumination chamber 2a in which the disinfection of the writing instrument 4a occurs.

The edge of the illumination chamber 2a facing the cover 5a is provided with a seal 9a such that no UV or UVC light can escape to the outside when the light sources 3a are activated.

Of course, the disinfection of the writing instrument 4a is no use if all users of the writing instrument 4a must unfold the cover 5a by hand.

Therefore, the device according to the second aspect of the invention provides that the cover 5a has an automatic opening and closing mechanism 6a. Several embodiments were described above to this end. Here, the principle operating on the basis of a clock timer is shown.

The housing 1a has a time switch in a controller 8a which deactivates the UV light sources 3a after a predeterminable time of the UV light irradiation of the writing instrument 4a and generates a signal for opening the illumination chamber 2a of the housing 1a such that the writing instrument 4a is accessible.

Thus, in this embodiment, only such writing instruments 4a are accessible to the user
which are disinfected and
when the UV or the UVC light is switched off.

The latter occurs before the opening of the cover 5a.

A rest 7a serves to close the illumination chamber 2a, said rest being adapted to be a pressure switch and activating the closing mechanism 6a upon placing the writing instrument 4a.

If the cover 5a is closed, the disinfection with UV or UVC light starts, controlled via the time switch of the controller 8a.

The disinfection can be sped up if the illumination chamber 2a is equipped with UV light-reflecting means.

Once the set disinfection time has expired, the light sources 3a are deactivated and then the automatic opening and closing mechanism 6a for opening the cover 5a is activated.

Considering the fact that the disinfection time is less than one minute long, no further device is necessary in order to, so to speak, pass the time during disinfection of another device.

FIG. 4 shows a tub-like housing 1a for accommodating a writing instrument 4a. The housing 1a is, inter alia, characterized by a foldable and/or rollable and/or insertable/removable cover 5b which can be a folding and/or sliding and/or slat door and can close the housing. The inner tub walls of the housing 1a are supplied with light sources 3a which can radiate UV light or UVC light and thereby form an illumination chamber 2a in which the disinfection of the writing instrument 4a occurs.

The edge of the illumination chamber 2a facing the cover 5b is optionally provided with a seal 9a such that no UV or UVC light can escape to the outside when the light sources 3a are activated.

Of course, the disinfection of the writing instrument 4a is no use if all users of the writing instrument 4a must push open/peel up/roll out the cover 5b by hand.

Therefore, the device according to the second aspect of the invention provides that the cover 5b has an automatic opening and closing mechanism 6a. Several embodiments were described above to this end. Here, the principle operating on the basis of a clock timer (8a) is shown.

The housing 1a has a time switch in a controller 8a which deactivates the UV light sources 3a after a presettable time of the UV light irradiation of the writing instrument 4a and generates a signal for opening the illumination chamber 2a of the housing 1a such that the writing instrument 4a is accessible.

Thus, in this embodiment, only such writing instruments 4a are accessible to the user
which are disinfected and
when the UV or the UVC light is switched off.

The latter occurs before the opening of the cover 5b.

A rest 7a serves to close the illumination chamber 2a, said rest being adapted to be a pressure switch and activating the closing mechanism 6a upon placing the writing instrument 4a.

If the cover 5b is closed, the disinfection with UV or UVC light starts, controlled via the time switch of the controller 8a.

The disinfection can be sped up if the illumination chamber 2a is equipped with UV light-reflecting means.

Once the set disinfection time has expired, the light sources 3a are deactivated and then the automatic opening and closing mechanism 6a for opening the cover 5b is activated.

Considering the fact that the disinfection time is less than one minute long, no further device is necessary in order to, so to speak, pass the time during disinfection of another device.

FIG. 4A shows the open state of the cover 5b and FIG. 4B shows the closed state of the cover 5b.

FIG. 5 shows a tub-like housing 1a for accommodating a writing instrument 4a. The housing 1a does not presently comprise a cover, but may do so. The inner tub walls of the housing 1a are supplied with light sources 3a which can radiate UV light or UVC light and thereby form an illumination chamber 2a in which the disinfection of the writing instrument 4a occurs.

As no cover is present in this case, users of the writing instrument 4a also need not push open/peel up/roll out a cover.

Therefore, the device according to the second aspect of the invention according to FIG. 5 provides that the device of the present invention additionally comprises a removable and/or insertable device (7a), more preferably a telescopic removable and/or insertable rod device (7a) which may be electronically and/or mechanically removed and which, like an elevator, can move upward and/or downward and/or laterally inward and/or outward therewith in order to move the comprised writing instrument 4a into the illumination chamber 2a and/or out of the illumination chamber 2a, preferably being electronically controlled (8b) and the light sources (3a) for the UV light only being activatable with a writing instrument (4a) at least partially, preferably entirely, inserted in the illumination chamber (2a) and deactivatable with the writing instrument (4a) at least partially, preferably entirely, removed from the illumination chamber (2a).

The housing 1a has a time switch in a controller 8a which deactivates the UV light sources 3a after a predeterminable time of the UV light irradiation of the writing instrument 4a (in the inserted state of the device; see FIG. 5a) and generates a signal for removing the writing instrument 4a from the illumination chamber 2a such that the writing instrument 4a is accessible; see FIG. 5B.

Thus, in this embodiment, only such writing instruments 4a are accessible to the user
which are disinfected and
when the UV or the UVC light is switched off.

The latter occurs before the removal of the writing instrument.

A rest 7a serves to activate the UV illumination in the illumination chamber 2a, said rest being adapted to be, e.g., a pressure switch and activating the inserting mechanism of the device, e.g. via an electrical motor 8b, preferably the telescopic rod device 10a, upon placing/resting the writing instrument 4a.

Once the writing instrument is at least partially, preferably entirely, inserted in the illumination chamber 2a, the disinfection with UV or UVC light starts, controlled via the time switch of the controller 8a; optionally, this can also be combined with the closing of an additional cover.

The disinfection can be sped up if the illumination chamber 2a is equipped with UV light-reflecting means.

Once the set disinfection time has expired, the light sources 3a are deactivated and then the automatic removal mechanism is activated; see FIG. 5B.

Considering the fact that the disinfection time is less than one minute long, no further device is necessary in order to, so to speak, pass the time during disinfection of another device.

FIG. 5A shows the inserted or mid-insertion state of the writing instrument 4a (see arrows) and FIG. 5B shows the removed or mid-removal state of the writing instrument 4a (see arrows).

The proposed device according to the second aspect of the invention operates safely, as, on the one hand, disinfection is possible and, on the other, any contact of the human body with UV or UVC light is eliminated.

### CONTINUED LIST OF REFERENCE NUMERALS

1a Housing
2a Illumination chamber
3a light sources
4a Writing instrument
5a cover
5b folding or sliding door and/or slat door as a foldable or rollable cover
6a automatic opening and closing mechanism
7a rest
8a controller
8b Controller device, preferably electronic and/or mechanical, for inserting and/or removing the removable device 10a, comprising the writing instrument 4a
9a seal
10a removable and/or insertable device, more preferably a telescopic removable and/or insertable rod device which may be electronically and/or mechanically removed and which, like an elevator, can move upward and/or downward and/or laterally inward and/or outward therewith in order to move the comprising writing instrument 4a into the illumination chamber 2a and/or out of the illumination chamber 2a, preferably being electronically controlled (8b) and the light sources (3a) for the UV light only being activatable with a writing instrument (4a) at least partially, preferably entirely, inserted in the illumination chamber (2a) and deactivatable with the writing instrument (4a) at least partially, preferably entirely, removed from the illumination chamber (2a).

In consideration of the aforementioned full disclosure of the second aspect of the invention, said aspect further comprises the embodiments as numbered in the following:

14. A device for accommodating and disinfecting a writing instrument (4a), comprising at least a housing (1a) having an illumination chamber (2a) which is illuminatable with UV light such that the entire outer surface of the writing instrument (4a) placed or inserted in the illumination chamber (2a) through an opening is irradiated, wherein the opening for placing the writing instrument (4a) comprises a cover (5a), characterized in that

the illumination chamber (2a) is completely closed by the closed cover (5a) and the light sources (3a) for the UV light are only activatable when the illumination chamber (2a) is completely closed and deactivatable upon or before opening the illumination chamber (2a).

15. The device according to Embodiment 14, characterized in that

the cover (5a) is a foldable or slidable or peelable lid or a blind which has an automatic opening and closing mechanism (6a) which is touchlessly activatable.

16. The device according to Embodiment 15, characterized in that

for touchless actuation of the cover (5a), a movement sensor is arranged on the housing (1a) which, upon registering a movement, generates a signal for actuating the automatic opening and closing mechanism (6a) .

17. The device according to Embodiment 15, characterized in that

for touchless actuation of the cover (5a), an insertion slot for any plastic card having a chip card format is provided in the housing (1a), wherein the inserting or removing of the plastic card is detectible and a signal for opening or closing the cover (5a) is triggerable therethrough.

18. The device according to any one of Embodiments 15 to 17, characterized in that

the housing (1a) has a time switch in a controller (8a) which deactivates the UV light sources (3a), preferably UVC light sources, after a predeterminable time of the UV light irradiation of the writing instrument (4a) and generates a signal for opening the illumination chamber (2a) of the housing (1a) such that the writing instrument (4a) is accessible.

19. The device according to any one of Embodiments 14 to 18, characterized in that

the cover (5a) and/or the rest surface of the cover (5a) are equipped with a seal (9a) on the housing (1a).

20. The device according to any one of Embodiments 14 to 19, characterized in that

the closing of the illumination chamber (2a) is coupled with the placing of the writing instrument (4a) such that a signal for closing the cover (5a) is generatable upon placing.

21. The device according to any one of Embodiments 14 to 20, characterized in that

the illumination of the illumination chamber (2a) with UV light, preferably UVC light, occurs directly via the light sources (3) and/or through illumination slits which are placed in front of the light sources (3) .

22. The device according to any one of Embodiments 14 to 21, characterized in that

the illumination chamber (2a) is at least partially lined or coated with UV light-reflecting means.

23. The device according to any one of Embodiments 14 to 22, characterized in that

the illumination chamber (2a) comprises a cover (5b) which is characterized by a sliding door and/or folding door and/or slat door configuration and is thus foldable and/or rollable, preferably electronically and/or mechanically foldable and/or rollable.

24. A device for accommodating and disinfecting a writing instrument (4a), comprising at least a housing (1a) having an illumination chamber (2a) which is illuminatable with UV light such that the entire outer surface of the writing instrument (4a) placed or inserted or put in the illumination chamber (2a) through an opening, wherein the opening for placing the writing instrument (4a) may optionally additionally comprise a cover (5a), characterized in that

the device further comprises a removable and/or insertable device, more preferably a telescopic removable and/or insertable rod device which may be electronically and/or mechanically removed and which, like an elevator, can move upward and/or downward and/or laterally inward and/or outward therewith in order to move the comprised writing instrument 4a into the illumination chamber 2a and/or out of the illumination chamber 2a, preferably being electronically controlled (8b) and the light sources (3a) for the UV light only being activatable with a writing instrument (4a) at least partially, preferably entirely, inserted in the illumination chamber (2a) and deactivatable with the writing instrument (4a) at least partially, preferably entirely, removed from the illumination chamber (2a).

25. An illumination chamber (2a) for accommodating and disinfecting a writing instrument (4a) with UV light, preferably UVC light, wherein the entire outer surface of the writing instrument (4a) is irradiated with UV light, preferably UVC light, characterized in that

for irradiating the outer surface of the writing instrument (4a), illumination of the illumination chamber (2a) with UV light, preferably UVC light, occurs directly via the light sources (3a) and/or through illumination slits which are placed in front of the light sources (3a).

26. The illumination chamber (2a) according to Embodiment 23, characterized in that

the illumination chamber (2a) is at least partially lined or coated with UV light-reflecting means.

1. A device for accommodating and disinfecting a writing instrument (4), comprising at least a housing (1) having a chamber with at least one opening for placing or inserting the writing instrument (4) in the chamber, characterized in that
the chamber is an illumination chamber (2) equipped with UV light sources (3) that irradiate the entire outer surface of the placed or inserted writing instrument (4).

2. The device according to claim 1, characterized in that
the illumination chamber (2) is formed to be a trough-shaped recess or trough or to be a bore or a blind hole.

3. The device according to claim 1 or 2, characterized in that
the UV light sources (3) emit UVC light.

4. The device according to any one of claims 1 to 3, characterized in that
centering elements (5) for the writing element (4) are arranged in the illumination chamber (2).

5. The device according to any one of claims 2 to 4, characterized in that
the illumination chamber (2) formed to be a trough-shaped recess or trough comprises a depth which is greater than the greatest cross-section of the writing instrument (4) and, in the case of a bore or a blind hole, the depth is greater than the length of the writing instrument (4).

6. The device according to any one of claims 1 to 5, characterized in that
a cover is arranged above the opening for placing the writing instrument (4) or a partial cover (8) is arranged spaced apart from the opening, said partial cover also being fitted with UV light sources (3) on the inside.

7. The device according to claim 6, characterized in that
the cover or the partial cover is arranged to be unfoldable or slidable or peelable.

8. A device for accommodating and disinfecting a writing instrument (4a), comprising at least a housing (1a) having an illumination chamber (2a) which is illuminatable with UV light such that the entire outer surface of the writing instrument (4a) placed or inserted in the illumination chamber (2a) through an opening is irradiated, wherein the opening for placing the writing instrument (4a) comprises a cover (5a), characterized in that
the illumination chamber (2a) is completely closed by the closed cover (5a) and the light sources (3a) for the UV light are only activatable when the illumination chamber (2a) is completely closed and deactivatable upon or before opening the illumination chamber (2a).

9. The device according to claim 8, characterized in that
the cover (5a) is a foldable or slidable or peelable lid or a blind which has an automatic opening and closing mechanism (6a) which is touchlessly activatable.

10. The device according to claim 9, characterized in that
for touchless actuation of the cover (5a), a movement sensor is arranged on the housing (1a) which, upon registering a movement, generates a signal for actuating the automatic opening and closing mechanism (6a).

11. The device according to any one of claims 9 to 10, characterized in that
the housing (1a) has a time switch in a controller (8a) which deactivates the UV light sources (3a) after a predeterminable time of the UV light irradiation of the writing instrument (4a) and generates a signal for opening the illumination chamber (2a) of the housing (1a) such that the writing instrument (4a) is accessible.

12. The device according to any one of claims 8 to 11, characterized in that
the closing of the illumination chamber (2a) is coupled with the placing of the writing instrument (4a) such that a signal for closing the cover (5a) is generatable upon placing.

13. The device according to any one of claims 8 to 12, characterized in that
the illumination chamber (2a) is at least partially lined or coated with UV light-reflecting means.

14. The device according to any one of claims 8 to 13, characterized in that
the illumination chamber (2a) comprises a cover (5b) which is characterized by a sliding door and/or folding door and/or slat door configuration and is thus foldable and/or rollable, preferably electronically and/or mechanically foldable and/or rollable.

15. A device for accommodating and disinfecting a writing instrument (4a), comprising at least a housing (1a) having an illumination chamber (2a) which is illuminatable with UV light such that the entire outer surface of the writing instrument (4a) placed or inserted or put in the illumination chamber (2a) through an opening, wherein the opening for placing the writing instrument (4a) may optionally additionally comprise a cover (5a), characterized in that
the device further comprises a removable and/or insertable device, more preferably a telescopingly removable and/or insertable rod device which may be electronically and/or mechanically removed and which, like an elevator, can move upward and/or downward and/or laterally inward and/or outward therewith in order to move the comprised writing instrument 4a into the illumination chamber 2a and/or out of the illumination chamber 2a, preferably being electronically controlled (8b) and the light sources (3a) for the UV light only being activatable with a writing instrument (4a) at least partially, preferably entirely, inserted in the illumination chamber (2a) and deactivatable with the writing instrument (4a) at least partially, preferably entirely, removed from the illumination chamber (2a).
